(19) United States
(12) Patent Application Publication
(10) Pub. No.: US 2014/0236032 A1
(43) Pub. Date: Aug. 21, 2014

(54) IMPLANTABLE HEART MONITORING DEVICE

(71) Applicant: BIOTRONIK SE & CO. KG, Berlin (DE)

(72) Inventors: GARTH GARNER, TIGARD, OR (US); J. CHRISTOPHER MOULDER, PORTLAND, OR (US); HANNES KRAETSCHMER, Portland, OR (US); R. HOLLIS WHITTINGTON, PORTLAND, OR (US); DIRK MUESSIG, WEST LINN, OR (US); SWETHA VENNELAGANTI, LAKE OSWEGO, OR (US)

(73) Assignee: BIOTRONIK SE & CO. KG, Berlin (DE)

(21) Appl. No.: 14/181,579

(22) Filed: Feb. 14, 2014

Related U.S. Application Data
(60) Provisional application No. 61/766,139, filed on Feb. 19, 2013.

(51) Int. Cl. A61B 5/0464; A61B 5/042

(52) U.S. Cl. CPC A61B 5/0464; A61B 5/042
USPC 600/515; 600/509

(57) ABSTRACT

An implantable heart monitoring device including a sensing unit, a signal quality analysis unit, and an evaluation unit. The sensing unit is connected to at least one electrode that picks up electric potentials. The sensing unit processes electrical signals corresponding to the electric potentials, and generates sense signals including events corresponding to myocardial contractions. The signal quality analysis unit determines whether a noise condition (NC) and/or a low signal indication is present for an event and generates an indication signal. The evaluation unit evaluates the sense signals and the indication signals, treats a detected event as non-valid if a NC and/or low signal indication signal is present for that event, and uses only intervals defined by two consecutive events which do not have a NC and/or a low signal indication to determine a rate of events of the sense signal.

This application claims the benefit of U.S. Provisional Patent Application 61/766,139, filed on 19 Feb. 2013, the specification of which is hereby incorporated herein by reference.

### BACKGROUND OF THE INVENTION

1. Field of the Invention

At least one embodiment of the invention relates to an implantable heart monitoring device.

2. Description of the Related Art

Typically, an implantable heart monitoring device may be a standalone device or part of an implantable heart stimulator such as dual-chamber (RA-RV), three-chamber (BiA-RV, or RA-BiV), or four-chamber (BiA-BiV) implantable cardiac devices, including pacemakers, defibrillators and cardioverters, which monitor and, if needed, stimulate cardiac tissue electrically to control the patient's heart rhythm.

Implantable heart stimulators, generally, may be used for cardiac rhythm management (CRM) for treating a variety of heart functional and rhythm disorders including but not limited to bradycardia, tachycardia or fibrillation, by way of electric stimulation pulses delivered to the heart tissue, such as the myocardium. A sufficiently strong stimulation pulse outside a heart chamber's refractory period typically leads to excitation of the myocardium of that heart chamber, which in turn is typically followed by a contraction of the respective heart chamber.

Tachycardia is a rhythm disorder that generally comprises a high cardiac rate while bradycardia generally refers to too low a heart rate. Fibrillation, typically, is a state wherein the heart myocytes are depolarizing in a non-coordinated manner, preventing the heart from pumping blood.

Depending on the disorder to be treated, such heart stimulators typically generate electrical stimulation pulses that are delivered to the heart tissue (myocardium) of a respective heart chamber according to an adequate timing regime. Delivery of stimulation pulses to the myocardium is usually achieved by means of an electrode lead that is typically electrically connected to a stimulation pulse generator inside a heart stimulator's housing and carries a stimulation electrode in the region of its distal end. A stimulation pulse is also called a pace. Similarly, pacing a heart chamber generally means stimulating a heart chamber by delivery of a stimulation pulse.

In order to be able to sense the contraction of a heart chamber, which occurs naturally without artificial stimulation and which is called an intrinsic contraction, the heart stimulator usually includes at least one sensing stage that is connected to a sensing electrode and the electrode is placed in or near the heart chamber. An intrinsic excitation of a heart chamber generally results in characteristic electrical potentials that may be picked up via the sensing electrode and may be evaluated by the sensing stage in order to determine whether an intrinsic excitation, or intrinsic event, has occurred.

Usually, a heart stimulator features separate stimulation pulse generators for each heart chamber to be stimulated. Therefore, in a dual chamber pacemaker, usually an atrial and a ventricular stimulation pulse generator for generating atrial and ventricular stimulation pulses are provided. Delivery of an atrial or a ventricular stimulation pulse causing an artificial excitation of the atrium or the ventricle, respectively, is called an atrial stimulation event AP (atrial paced event) or a ventricular stimulation event VP (ventricular paced event), respectively.

Similarly, common heart stimulators generally feature separate sensing channels for each heart chamber to be of interest. In a dual chamber pacemaker, usually two separate sensing channels, an atrial sensing channel and a ventricular sensing channel, are provided that are capable of detecting intrinsic atrial events AS (atrial sensed event) or intrinsic ventricular events VS (ventricular sensed event), respectively.

In a heart cycle, an excitation of the myocardium typically leads to a depolarization of the myocardium that leads to a contraction of the heart chamber. If the myocardium is fully depolarized, it is typically not susceptible to further excitation and is thus refractory. Thereafter, the myocardium typically repolarizes and thus relaxes and the heart chamber expands again. In a typical intracardiac electrogram (IEGM), depolarization of the ventricle corresponds to a signal known as the “R-wave”. The “R-wave” is commonly preceded by a downward deflection, known as the “Q-wave” and followed by another downward deflection, known as the “S-wave”. Normal “Q-waves” represent depolarization of the interventricular septum. Any combination of Q-wave, R-wave, or S-wave is generally called a QRS complex. The repolarization of the ventricular myocardium typically coincides with a signal known as the “T-wave”. Atrial depolarization is generally manifested by a signal known as the “P-wave”.

In a healthy heart, initiation of the cardiac cycle normally begins with depolarization of the sinoatrial (SA) node. This specialized structure is located in the upper portion of the right atrium wall and acts as a natural “pacemaker” of the heart. In a normal cardiac cycle and in response to the initiating SA depolarization, the right atrium generally contracts and forces the blood that has accumulated therein into the ventricle. The natural stimulus causing the right atrium to contract is typically conducted to the right ventricle via the atrioventricular node (AV node) with a short, natural delay, the atrioventricular delay (AV-delay). Thus, a short time after the right atrial contraction (a time sufficient to allow the bulk of the blood in the right atrium to flow through the one-way valve into the right ventricle), the right ventricle typically contracts, forcing the blood out of the right ventricle to the pulmonary artery. A typical time interval between contraction of the right atrium and contraction of the right ventricle may be 100 ms, and a typical time interval between contraction of the right ventricle and the next contraction of the right atrium may be 800 ms. Thus, it is generally a right atrial contraction (A), followed a relatively short time thereafter by a right ventricle contraction (V), followed a relatively long time thereafter by the next right atrial contraction, that produces the desired AV synchrony. Where AV synchrony exists, the heart typically functions very efficiently as a pump in delivering life-sustaining blood to body tissue, and where AV synchrony is absent, the heart typically functions as an inefficient pump, largely because the right ventricle is contracting when it is not filled with blood.

Similarly, the left ventricle generally contracts in synchrony with right atrium and the right ventricle with a positive or negative time delay between a right ventricular contraction and a left ventricular contraction.

A pacemaker generally induces a contraction of a heart chamber by delivery of a stimulation pulse (pacing pulse) to the chamber when no natural (intrinsic) contraction of the chamber occurs in due time. A contraction of a heart chamber is often called an “event.” Because a contraction may be an intrinsic contraction, which may be sensed by a sensing stage of a pacemaker, such an event is typically called a sensed event. A contraction due to delivery of a stimulation pulse is generally called a paced event. A sensed event in the atrium is typically called AS, and a paced atrial event is typically called AP. Similarly, a sensed event in the ventricle is typically called VS and a paced ventricular event is typically called VP.

To mimic the natural behavior of a heart, a dual-chamber pacemaker generally provides for an AV-delay timer to provide for an adequate time delay (atrioventricular delay, AV-delay, AVD) between a natural (intrinsic) or a stimulated (paced) right atrial contraction and a right ventricular contraction. In a similar way, a biventricular pacemaker typically provides for an adequate time delay (VV-delay, VVD) between a right ventricular contraction and a left ventricular contraction.

The time delay for a left ventricular (stimulated, paced) contraction may generally be timed from a scheduled right ventricular contraction which has not yet occurred or from a natural (intrinsic) or a stimulated (paced) right atrial contraction. In the latter case, a left ventricular stimulation pulse is typically scheduled by a time interval AVD+VVD.

To deal with possibly occurring natural (intrinsic) atrial or ventricular contractions, a demand pacemaker generally schedules a stimulation pulse for delivery at the end of the AV-delay or the VV-delay, respectively. The delivery of the stimulation pulse is generally inhibited, if a natural contraction of the heart chamber to be stimulated is sensed within the respective time delay.

A natural contraction of a heart chamber may generally be similarly detected by evaluating the electrical signals sensed by the sensing channels. In the sensed electrical signal, the depolarization of an atrium muscle tissue is typically manifested by occurrence of a P-wave. Similarly, the depolarization of ventricular muscle tissue is typically manifested by the occurrence of an R-wave. The detection of a P-wave or an R-wave generally signifies the occurrence of intrinsic atrial, AS, or ventricular, VS events, respectively.

A dual chamber pacemaker, featuring an atrial and a ventricular sensing stage, and an atrial and a ventricular stimulation pulse generator, may be generally operated in a number of stimulation modes such as VVI, AAI, or DD. Typically in VVI, atrial sense events are ignored and no atrial stimulation pulses are generated, but only ventricular stimulation pulses are delivered in a demand mode. In AAI, typically, ventricular sense events are ignored and no ventricular stimulation pulses are generated, but only atrial stimulation pulses are delivered in a demand mode. In DDD, typically both atrial and ventricular stimulation pulses are delivered in a demand mode. In such a DDD mode of pacing, ventricular stimulation pulses may generally be generated in synchrony with sensed intrinsic atrial events and thus in synchrony with an intrinsic atrial rate, wherein a ventricular stimulation pulse is scheduled to follow an intrinsic atrial contraction after an appropriate atrioventricular delay (AV-delay; AVD), thereby maintaining the hemodynamic benefit of atrioventricular synchrony.

To allow for correct diagnosis with an implantable cardiac monitoring device and for an effective stimulation with an implantable heart stimulator, the sensing stage of the implantable cardiac monitoring device or the implantable heart stimulator may typically identify if a heart functional or rhythm disorder is present in the heart beat.

For example, WIPO Patent Publication 2012/015498 A1 to Stadler et al., entitled “Prevention of False Asystole or Bradycardia Detection”, appears to disclose a medical system and method to reject undersensing in a signal indicative of cardiac activity, e.g., ECG. The medical system of Stadler et al. detects at least one of a asystole or a bradycardia based on the comparison of the amplitude of the signal to a first threshold. According to Stadler et al., the medical system may determine whether the detection of the asystole or the bradycardia is false based on the comparison of an amplitude of a detected R-wave in the signal to at least a second threshold.

European Patent 1 237 622 B1 to Lin et al., entitled “An Automatic External Cardioverter/Defibrillator with Cardiac Rate Detector”, appears to disclose an external defibrillator with an electrode, a sense circuit, a cardiac arrhythmia detector, a microprocessor-based controller, and a therapy delivery circuit is presented. The electrode is coupled externally to a body, the sense circuit is coupled to the electrode to sense a physiological signal indicative of intrinsic cardiac activity, and the cardiac arrhythmia detector is coupled to the sense circuit to detect a cardiac arrhythmia based on the physiological signal. In addition, the cardiac detector appears to include a rate detector, which detects a first average intrinsic cardiac rate for a predetermined number N of cardiac events and a second average by dropping measurements related to one of the events, with the second average being taken for the remaining N−1 cardiac events. According to Lin et al., the therapy delivery circuit delivers electrical therapy pulses to a patient to correct abnormal cardiac arrhythmia.

### BRIEF SUMMARY OF THE INVENTION

It is an object of at least one embodiment of the invention to provide an improved apparatus and a method for monitoring electric potentials corresponding to myocardial contraction, i.e., a cardial beat or heartbeat.

At least one embodiment of the invention includes an implantable heart monitoring device including one or more of a sensing unit, a signal quality analysis unit, and an evaluation unit. The sensing unit, in one or more embodiments, may be connected to at least one electrode. The at least one electrode, in at least one embodiment, may pick up electric potentials. According to one or more embodiments, the sensing unit may process electrical signals corresponding to the electric potentials, to detect signal components representing a myocardial contraction, and may generate sense signals representing detected events corresponding to myocardial contractions. By way of at least on embodiment, the signal quality analysis unit may determine whether a noise condition (NC), a low signal indication or both are present for a detected event, and may generate a noise condition and/or a low signal indication signal. The evaluation unit, in one or more embodiments, may be connected to the sensing unit and the signal quality analysis unit, and may evaluate sense signals and noise condition, and/or low signal indication signals. The evaluation unit in at least one embodiment may be further configured to treat a detected event as non-valid or invalid, respectively, if a noise condition (NC) and/or low signal indication signal is present for that event, and may use only intervals defined by two consecutive events which do not have a noise condition (NC) and/or a low signal indication to determine a rate of events of the sense signal.

By way of at least one embodiment, low signal quality due to noise, or due to a low signal strength, may affect the reliability of the sensing of events, in particular with respect to the moment of occurrence of the event. An invalid event may be an event that has an associated noise condition (NC) or low signal indication. An interval defined by an invalid event may have a large error, in one or more embodiments, as the moment of occurrence of the invalid event may not be defined with high certainty, leading to large errors for interval durations and therefore these intervals may be unusable for the determination of a rate of events, i.e., unusable or unused intervals.

In at least one embodiment, rhythm classification may equally be based on an analysis of interval duration or an analysis of rate of events, because an interval may be the inverse of a rate of events.

According to one or more embodiments, the signal quality analysis unit may be an integral part of the sensing unit, thus forming a combined sensing/signal quality analysis unit that may detect signal components that may represent cardiac events, and may generate a sense signal, e.g., a marker signal and that may flag a sense signal as noisy signal or low signal if a noise condition or a low signal indication is detected, respectively. The combined sensing and signal quality analysis unit, in at least one embodiment, may also be configured to generate a flag only for the events with a noise condition and/or a low signal indication with respect to an individual sense signal. Alternatively, in at least one embodiment, the signal quality analysis unit may be implemented as a separate unit or may be part of the evaluation unit.

By way of one or more embodiments, the evaluation unit may treat the sense signals or events for which a noise condition or a low signal indication was detected as “invalid” sense signals or “invalid” events. In at least one embodiment, the evaluation unit may classify the events of sense signals for which a noise condition or a low signal condition was detected as “invalid” events. In this way, high ventricular rate, bradycardia, and asystole may be detected with a high specificity.

In at least one embodiment, the sensing unit of the implantable heart monitoring device may be a ventricular sensing unit that may detect ventricular events.

The evaluation unit, according to one or more embodiments, may execute various monitoring and stimulation algorithms. In at least one embodiment, the algorithms may be executed in parallel or serially. The evaluation unit, in embodiments of the invention, may use parameters, e.g., rate of events determined from used intervals of the sense signal, interval duration, or other parameters, determined from a combined sense signal from all electrodes for all algorithms executed in parallel. Alternatively, in one or more embodiments, the evaluation unit may also determine individual parameters, e.g., from sense signals from different electrodes, to be used for different algorithms. As an example, an evaluation unit in embodiments of the invention may run a monitoring algorithm for each of two ventricles of the heart, with parameters determined from a left ventricle for the monitoring algorithm of the left ventricle and parameters determined from a right ventricle for the monitoring algorithm of the right ventricle.

Preferably, in one or more embodiments, the evaluation unit of the implantable heart monitoring device may detect a high ventricular rate, if a high rate detection counter exceeds a predetermined threshold. In at least one embodiment, the evaluation unit may increment the high rate detection counter each time the determined rate of events in a used interval exceeds a predetermined threshold of a high ventricular rate detection limit. The used intervals may be defined by two consecutive events which do not have a noise condition (NC) and/or a low signal indication. According to one or more embodiments, the predetermined threshold of the high ventricular rate detection limit may be in the range from 150 to 200 bpm and is preferably adjustable in steps of 10 bpm. A default value for the predetermined threshold of the high ventricular rate detection limit may for example be 180 bpm. In at least one embodiment, the predetermined threshold of the high rate detection counter may be in the range from 4 to 16 and is preferably adjustable in steps of 4. A default value for the predetermined threshold of the high rate detection counter, in one or more embodiments, may for example be 8. By way of at least one embodiment, the evaluation unit may decrement the high rate detection counter each time the determined rate of events in a used interval is below the predetermined threshold of the high ventricular rate detection limit. The evaluation unit, in embodiments of the invention, may also be configured to decrement the high rate detection counter each time an event has a noise condition (NC) and/or a low signal indication associated with it, meaning an invalid event. In an alternative embodiment, the high rate detection counter remains unchanged for invalid events. The evaluation unit may also be configured to decrement the high rate detection counter for each time of occurrence of an unused interval, in at least one embodiment, which may be intervals that include at least one invalid event, or to remain unchanged for unused intervals. In one or more embodiments, the evaluation unit may execute a high ventricular rate detection algorithm to detect whether a high ventricular rate is present in the sense signals.

By way of at least one embodiment, the evaluation unit may detect a termination of the high ventricular rate if the high ventricular rate was detected and a termination counter exceeds a predetermined threshold. Alternatively, in one or more embodiments, the evaluation unit may also detect the termination of the high ventricular rate if the termination counter is equal to or greater than the predetermined threshold value of the termination counter. The predetermined threshold of the termination counter, in at least one embodiment, may be in the range from 4 to 16 and is preferably adjustable in steps of 1. A default value for the predetermined threshold of the termination counter may for example be 5. Preferably, in one or more embodiments, the evaluation unit may increment the termination counter each time the determined rate of events in a used interval may be below a predetermined threshold of the high ventricular rate detection limit. The predetermined threshold of the high ventricular rate detection limit, in at least one embodiment, may be different compared to the detection of a high ventricular rate. In at least one embodiment, the termination counter may be set to zero each time the determined rate of events in a used interval exceeds the predetermined threshold of the high ventricular rate detection limit.

Alternatively, in one or more embodiments, the termination counter may be decremented, e.g., by 1, 2, or another number, as long as the termination counter is above 0, each time the determined rate of events in a used interval exceeds the predetermined threshold of the high ventricular rate detection limit. The evaluation unit, in at least one embodiment, preferably executes a high ventricular rate termination detection algorithm to detect whether a high ventricular rate may be terminated in the sense signals.

In one or more embodiments, the evaluation may detect an asystole if a used interval is longer than a predetermined asystole interval limit. The predetermined asystole interval limit, in at least one embodiment, may be in the range from 2 seconds to 10 seconds and may preferably be adjustable in steps of 1 second. A default value of the predetermined asystole interval limit, in one or more embodiments, may for example be 3 seconds. In embodiments of the invention, the evaluation unit may preferably execute an asystole detection algorithm to detect whether an asystole may be present in the sense signals.

The evaluation unit in at least one embodiment may also detect bradycardia, if an average rate of events is less than a predetermined Brady rate limit for a predetermined bradycardia duration. The predetermined Brady rate limit, in one or more embodiments, may be in the range from 30 to 80 bpm and may preferably be adjustable in steps of 5 bpm. A default value of the Brady rate limit in embodiments of the invention may for example be 40 bpm. The predetermined bradycardia duration, according to at least one embodiment, may be in the range from 5 to 30 seconds and may be adjustable in steps of 5 seconds. A default value of the bradycardia duration, in embodiments of the invention, may be for example 10 seconds. The evaluation unit, in at least one embodiment, may preferably execute a Brady rate limit detection algorithm to detect whether bradycardia may be present in the sense signals.

According to one or more embodiments, the evaluation unit may find a minimum number of used intervals whose sum exceeds the predetermined bradycardia duration. The sum of the used intervals, in at least one embodiment, may be equal to or greater than the predetermined bradycardia duration. The evaluation unit, in one or more embodiments, may preferably determine an average duration of the used intervals in the bradycardia duration. Preferably, in one or more embodiments of the invention, the evaluation unit may convert the average duration into an average rate of events and may determine whether the average rate of events may be below the predetermined Brady rate limit. If the average rate of events of the sum of used intervals exceeding the predetermined bradycardia duration is below the predetermined Brady rate limit, bradycardia is detected, according to at least one embodiment. In one or more embodiments, the evaluation unit may add the newest used interval to the minimum number of used intervals when the evaluation unit may be in use and sense signals comprising events are received. In one or more embodiments, the oldest used interval may be removed from the minimum number of used intervals if the sum of the used intervals with the newest interval and without the oldest interval exceeds the predetermined bradycardia duration. In another embodiment, the evaluation unit may remove the oldest used interval from the minimum number of used intervals if an event has a noise condition (NC) and/or a low signal indication, i.e., if an invalid event is present in the sense signals.

In at least one embodiment, the evaluation unit may detect bradycardia, if an average rate of events decreases by a predetermined percentage threshold, i.e., detection of a Brady rate drop. A change in the average rate of events may be determined by comparing a pre-interval average comprising a predetermined pre-interval number of used intervals and a post-interval average comprising a predetermined post-interval number of used intervals, according to one or more embodiments of the invention. The predetermined pre-interval number may for example be 32, 48, or 64. In at least one embodiment, a default value for the predetermined pre-interval number may preferably be 48. The predetermined post-interval number may for example be 4, 8, or 16, wherein a default value for the predetermined post-interval number is preferably 8. In one or more embodiments, the evaluation unit may preferably execute a brady rate drop detection algorithm to detect whether bradycardia is present in the sense signals.

In at least one embodiment, the evaluation unit may set a number of useable intervals to the predetermined post-interval number, when bradycardia is detected. Following an invalid event, according to one or more embodiments, the oldest used interval may be removed from the number of used intervals and the number of useable intervals may be decremented by 1. In this case a newest interval may not be added to the sum of used intervals, as the newest interval may be unuseable.

The evaluation unit in at least one embodiment may preferably detect bradycardia, if Brady rate drop or Brady rate limit is detected. In at least one embodiment, both bradycardia detection algorithms, i.e., Brady drop rate detection algorithm and Brady rate limit detection algorithm, may preferably run in parallel on the evaluation unit. Once bradycardia is detected both bradycardia detection algorithms, i.e., in one or more embodiments, Brady drop rate detection algorithm and Brady rate limit, may switch to a bradycardia termination detection mode. In at least one embodiment, the Brady rate drop detection method may include the step of removing used intervals from the sum of used interval following a predetermined number of invalid events. The number of useable intervals, in one or more embodiments, may be set to 0 if an event has a noise condition (NC) and/or a low signal indication. The predetermined invalid event count may, for example, be 1, 2, 3 or 4, wherein a default value for the predetermined invalid event count may preferably be 1.

By way of one or more embodiments, the evaluation unit may detect a termination of bradycardia, if bradycardia was detected and a predetermined bradycardia termination counter exceeds a predetermined threshold. The bradycardia termination counter, in at least one embodiment, may preferably be set to zero when bradycardia is detected. Preferably, in embodiments of the invention, the bradycardia termination counter may be incremented for each used interval shorter than the interval equivalent of the predetermined Brady rate limit. The predetermined threshold of the Brady rate limit used for Brady rate limit detection may preferably be the same as the predetermined threshold used for termination of Brady rate limit and Brady rate drop, in one or more embodiments. The bradycardia termination counter may be decremented, in at least one embodiment, if the bradycardia termination counter is greater than zero and an invalid event is detected. By way of at least one embodiment, the bradycardia termination counter is preferably decremented by 1 for each event which has a noise condition (NC) and/or a low signal indication. The bradycardia termination counter, in one or more embodiment, may also be decremented by 2, 3 or another number, or the bradycardia termination counter may be set to zero. Preferably, in at least one embodiment, the bradycardia termination counter may be set to zero for each used interval that is longer than or equal to the interval equivalent of the predetermined Brady rate limit.

One or more embodiments may provide methods for detecting bradycardia, asystole, and/or high ventricular rate (HVR). The methods, in at least one embodiment, may preferably be integrated in monitoring algorithms, stimulating algorithms and/or monitoring and stimulating algorithms, which may be executed in parallel on the evaluation unit.

At least one embodiment of a high ventricular rate detection method may include one or more of the following steps:

Detect an event corresponding to a myocardial contraction, i.e. a heart beat.

Determine whether the event is a useable event or an invalid event. The invalid event is an event that comprises a noise condition and/or a low signal indication.

Determine a used interval if two consecutive useable events are present in the present interval.

Increment the high rate detection counter if the used interval exceeds a predetermined duration. The used intervals may include the intervals, which may be defined by two consecutive events that do not have a noise condition (NC) and/or a low signal indication.

Decrement the high rate detection counter if the used interval is below the predetermined duration.

Optionally the method may comprise the step of decrementing the high rate detection counter if an event has a noise condition (NC) and/or a low signal indication, meaning if the event is an invalid event.

Detect a high ventricular rate, if the high rate detection counter exceeds a predetermined threshold.

One or more embodiments of a high ventricular rate termination detection method may include one or more of the following steps:

Initialize the high ventricular rate termination detection method only if a high ventricular rate was detected and set a termination counter to zero.

Detect an event corresponding to a myocardial contraction, i.e., a heart beat.

Determine whether the event is a useable event or an invalid event.

Determine a used interval if two consecutive useable events are present in the present interval.

Increment the termination counter if the used interval is below a predetermined duration.

Set the termination counter to zero if the used interval exceeds the predetermined duration.

Detect a termination of the high ventricular rate, if the termination counter exceeds a predetermined threshold.

At least one embodiment of an asystole detection method may include one or more of the following steps:

Detect an event corresponding to a myocardial contraction.

Determine whether the event is a useable event or an invalid event.

Determine a used interval if two consecutive useable events are present in the present interval.

Detect an asystole if the used interval is longer than a predetermined asystole interval limit.

At least one embodiment of a Brady rate limit detection method may include one or more of the following steps:

Detect an event corresponding to a myocardial contraction.

Determine whether the event is a useable event or an invalid event.

Determine a used interval if two consecutive useable events are present in the present interval.

Add the used interval to a sum of intervals.

Determine whether the sum of intervals exceeds the predetermined bradycardia duration.

An optional step may be to remove the oldest interval, if the duration of the sum of used intervals without the oldest interval exceeds the predetermined bradycardia duration.

If the sum of intervals exceeds the predetermined bradycardia duration, determine an average duration of the summed used intervals.

Detect bradycardia, if the average interval is more than the predetermined duration.

At least one embodiment of the Brady rate limit detection method may include the step of removing the oldest used interval from the sum of used intervals, if an event has a noise condition (NC) and/or a low signal indication, i.e., if an invalid event is detected.

At least one embodiment of a Brady rate drop detection method may include one or more of the following steps:

Detect an event corresponding to a myocardial contraction.

Determine whether the event is a useable event or an invalid event.

Determine a used interval if two consecutive useable events are present in the present interval.

Add the used interval to a sum of intervals.

Determine whether the sum of intervals exceeds the sum of a pre-interval number of used intervals and a post-interval number of used intervals.

If the sum of intervals exceeds the sum of the pre-interval number of used intervals and the post-interval number of used intervals, determine a pre-interval average and a post-interval average.

Compare pre-interval average and post-interval average to determine a change in the average rate of events.

Detect bradycardia, if the average rate of events decreases by a predetermined percentage threshold, i.e., if the post-interval average is larger than a predetermined fractional of the pre-interval average.

At least one embodiment of the Brady rate drop detection method may include the step of removing the oldest used interval from the sum of used intervals, if an event has a noise condition (NC) and/or a low signal indication, i.e., if an invalid event is detected.

At least one embodiment of a bradycardia termination detection method may include one or more of the following steps:

Initialize bradycardia termination detection method only if bradycardia was detected and set a bradycardia termination counter to zero.

Detect an event corresponding to a myocardial contraction. Determine whether the event is a useable event or an invalid event.

Determine a used interval if two consecutive useable events are present in the present interval. Increment the bradycardia termination counter if the used interval is shorter than the interval equivalent of the predetermined Brady rate limit.

An optional step may be to decrement the bradycardia termination counter if an invalid event is detected and the bradycardia termination counter is greater than zero.

Set the bradycardia termination counter to zero if the used interval is longer than or equal to the interval equivalent of the predetermined Brady rate limit.

Detect termination of bradycardia if the predetermined bradycardia termination counter exceeds a predetermined threshold.

One or more embodiments of the Brady rate drop detection method may include the step of removing used intervals from the sum of used interval following a predetermined number of invalid events. In at least one embodiment, the number of useable intervals may be set to 0 if an event has a noise condition (NC) and/or a low signal indication. The predetermined invalid event count may, for example, be 1, 2, 3 or 4, wherein a default value for the predetermined invalid event count is preferably 1.

### BRIEF DESCRIPTION OF THE DRAWINGS

The above and other aspects, features and advantages of at least one embodiment of the invention will be more apparent from the following more particular description thereof, presented in conjunction with the following drawings wherein:

FIG. 1 shows a remote monitoring system and a heart stimulator according to one or more embodiments of the invention;

FIG. 2 illustrates a heart stimulator connected to electrode leads that are placed in a heart according to one or more embodiments of the invention;

FIG. 3 depicts a schematic block diagram of some components of the heart stimulator of FIG. 2 according to one or more embodiments of the invention;

FIG. 4 shows input and output interfaces of a high ventricular rate (HVR) according to one or more embodiments of the invention;

FIG. 5 shows a flow chart of a HVR algorithm for detection and termination of a HVR according to one or more embodiments of the invention;

FIG. 6 shows two intervals of an exemplary evaluation of a high ventricular rate (HVR) according to one or more embodiments of the invention;

FIG. 7 shows a first exemplary evaluation of a high ventricular rate (HVR) with HVR detection according to one or more embodiments of the invention;

FIG. 8 shows a first exemplary evaluation of a high ventricular rate (HVR) without HVR detection according to one or more embodiments of the invention;

FIG. 9 shows a first exemplary evaluation of a high ventricular rate (HVR) with HVR termination according to one or more embodiments of the invention;

FIG. 10 shows a second exemplary evaluation of a high ventricular rate (HVR) with HVR termination according to one or more embodiments of the invention;

FIG. 11 shows an example of a detection of an asystole according to one or more embodiments of the invention;

FIG. 12 shows a first exemplary evaluation of a Brady rate limit without detection of bradycardia according to one or more embodiments of the invention;

FIG. 13 shows a second exemplary evaluation of a Brady rate limit with an interval sum below a programmed duration according to one or more embodiments of the invention;

FIG. 14 shows a third exemplary evaluation of a Brady rate limit with an interval sum below a programmed duration according to one or more embodiments of the invention;

FIG. 15 shows a fourth exemplary evaluation of a Brady rate limit with an interval sum below a programmed duration according to one or more embodiments of the invention;

FIG. 16 shows a fifth exemplary evaluation of a Brady rate limit with an average rate of events indicative of bradycardia according to one or more embodiments of the invention;

FIG. 17 shows a first exemplary evaluation of a Brady rate drop with detection of bradycardia according to one or more embodiments of the invention;

FIG. 18 shows a second exemplary evaluation of a Brady rate drop with a too small number of intervals according to one or more embodiments of the invention;

FIG. 19 shows a third exemplary evaluation of a Brady rate drop with a too small number of intervals according to one or more embodiments of the invention; and

FIG. 20 shows a fourth exemplary evaluation of a Brady rate drop without detection of bradycardia according to one or more embodiments of the invention.

### DETAILED DESCRIPTION OF THE INVENTION

The following description is of the best mode presently contemplated for carrying out at least one embodiment of the invention. This description is not to be taken in a limiting sense, but is made merely for the purpose of describing the general principles of the invention. The scope of the invention should be determined with reference to the claims.

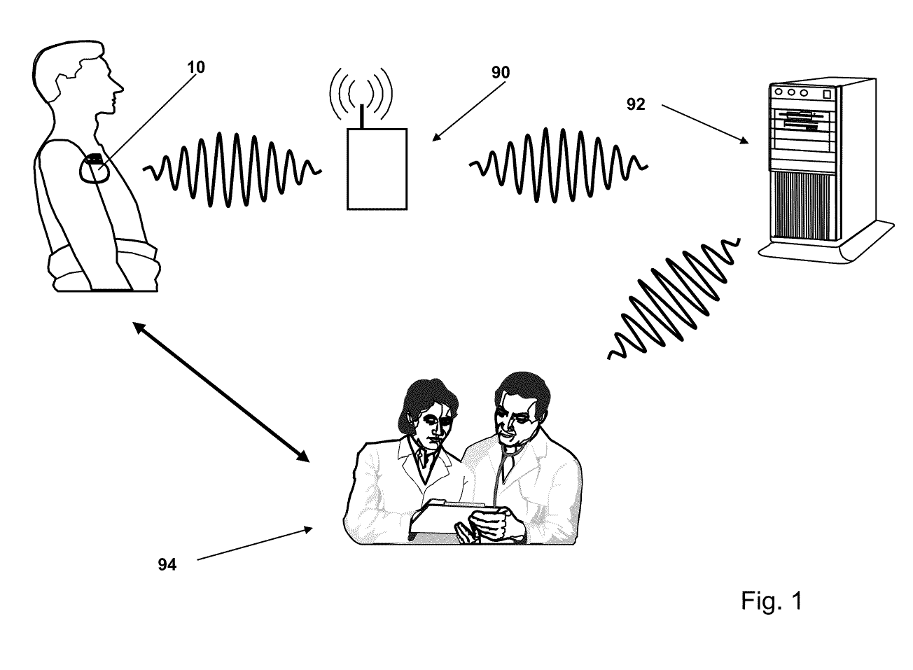

FIG. 1 shows a remote monitoring system and a heart stimulator according to one or more embodiments of the invention. As shown in FIG. 1, a remote monitoring system may include one or more of an implantable heart monitor or stimulator 10, an external device 90 and a central data server 92 of a central service center. Such a system, in at least one embodiment, may allow data communication between the implantable heart monitor and stimulator 10 and central server 92 via the external device 90. External device 90, in at least one embodiment, may communicate wirelessly with implantable heart monitor and stimulator 10.

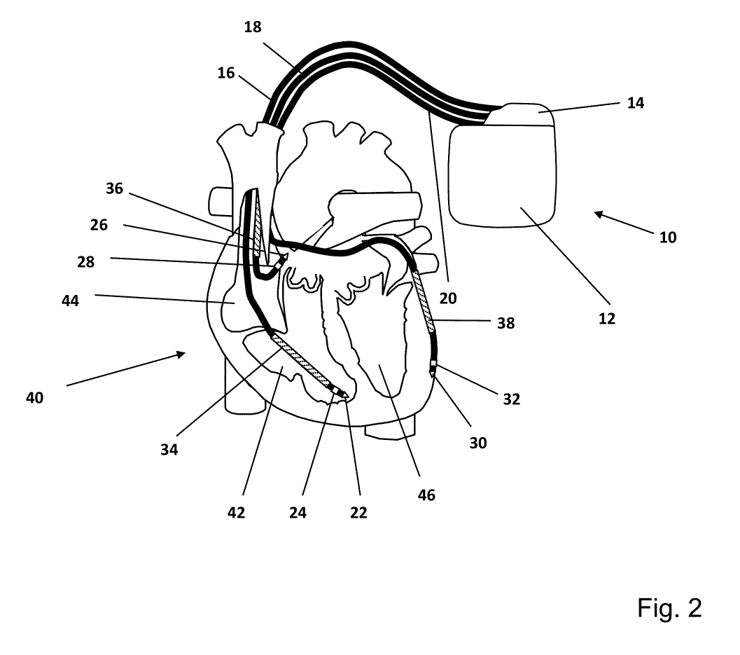

FIG. 2 illustrates a heart stimulator connected to electrode leads that are placed in a heart according to one or more embodiments of the invention. As shown in FIG. 2, the implantable heart monitor and stimulator 10 may include one or more of a housing or case 12 and a header 14.

In at least one embodiment, the implantable heart monitor and stimulator 10 may be connected to three electrode leads, namely a right ventricular electrode lead 16, a right atrial electrode lead 18 and a left ventricular electrode lead 20.

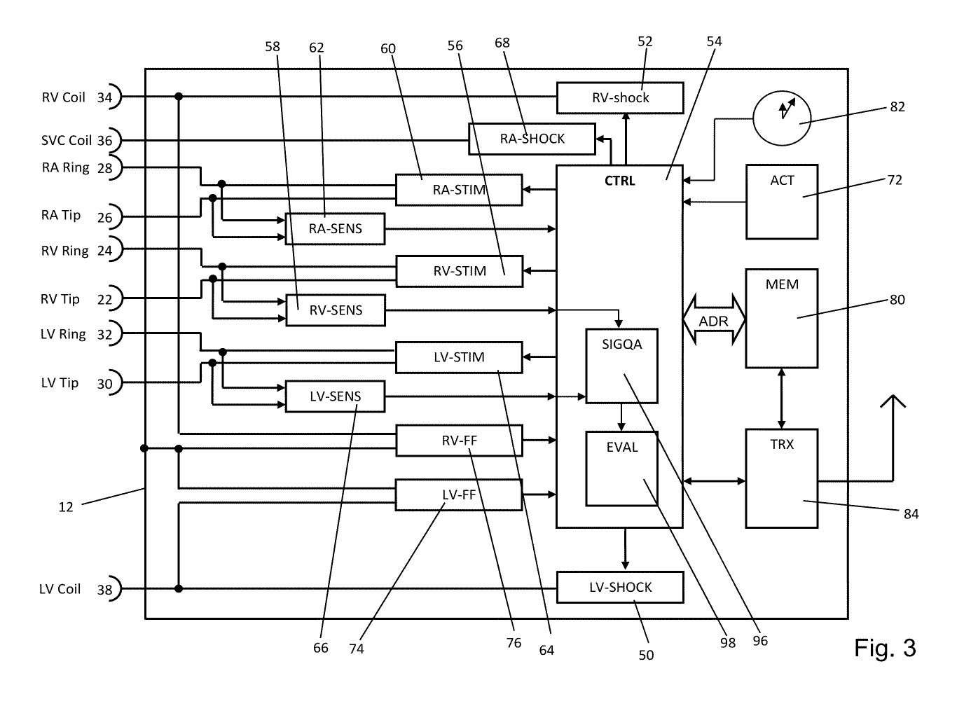

FIGS. 2 and 3 illustrate the pacing system that includes the implantable heart monitor and stimulator 10 and the connected leads 16, 18, and 20. In one or more embodiments, the right atrial electrode lead 18 may include one or more of a distal right atrial tip electrode 26 (RA-tip) at the distal end of right atrial electrode lead 18 and a proximal right atrial ring electrode 28 (RA-ring), as well as a superior vena cava coil electrode 36 (SVC-coil) that may have a large surface area.

In one or more embodiments, the right ventricular electrode lead 16 may include one or more of a distal right ventricular tip electrode 22 (RV-tip) at the distal end of right ventricular electrode lead 16 and a proximal right ventricular ring electrode 24 (RV-ring), as well as a right ventricular defibrillation coil electrode 34 (RV-coil) that may have a large surface area.

Similarly, according to at least one embodiment, the left ventricular (LV) lead may include one or more of a distal left ventricular tip electrode 30 (LV-tip) and a proximal left ventricular ring electrode 32 (LV-ring), as well as a defibrillation coil electrode 38 (LV-coil) that has large surface area. The left ventricular electrode lead 20, in at least one embodiment, may pass trough the coronary sinus of heart 40.

By way of one or more embodiments, each electrode and shock coil of electrode leads 16 to 20 may be separately connected to an electric circuit enclosed by case 12 of heart stimulator 10 by way of electrical contacts of a plug (not shown) at the proximal end of each electrode lead 16 to 20 and corresponding contacts (not shown) in header 14 of heart stimulator 10.

FIG. 3 depicts a schematic block diagram of some components of the heart stimulator of FIG. 2 according to one or more embodiments of the invention. As shown in FIG. 3, SVC shock coil 36 may be connected to right atrial shock generator 68 that may be controlled by a control unit 54 of heart stimulator 10.

Similarly, in at least one embodiment of the invention, right ventricular shock coil 34 may be connected to a right ventricular shock generator 52 that may be connected to control unit 54, and left ventricular shock coil 38 may be connected to a left ventricular shock generator 50 that may also be connected to control unit 54.

In one or more embodiments, right atrial tip electrode 26 and right atrial ring electrode 28 may both be connected to a right atrial stimulation pulse generator 60 and a right atrial sensing stage 62, that may internally both be connected to control unit 54.

By way of at least one embodiment, right atrial stimulation pulse generator 60 may generate atrial stimulation pulses of sufficient strength to cause an excitation of atrial myocardium by an electrical pulse delivered via right atrial tip electrode 26 and right atrial ring electrode 28. Preferably, in one or more embodiments, means may adapt the right atrial stimulation pulse strength to the stimulation threshold in the right atrium.

In at least one embodiment, right atrial sensing stage 62 may pick up myocardial potentials indicating an intrinsic atrial excitation that corresponds to a natural atrial contraction. In one or more embodiments, by way of right atrial sensing stage 62, the right atrium 44 of heart 40 in a demand mode may be stimulated, wherein a right atrial stimulation pulse may be inhibited if an intrinsic atrial event (intrinsic atrial excitation) is sensed by right atrial sensing stage 62 prior to expiration of an atrial escape interval.

In a similar manner, in one or more embodiments, right ventricular ring electrode 24 and right ventricular tip electrode 22 may be connected to right ventricular stimulation pulse generator 56 and to a right ventricular sensing stage 58 that in turn may be connected to control unit 54. The right ventricular sensing stage 58, in at least one embodiment, may be further connected to a signal quality analysis unit 96 of the control unit 54, which may determine whether a noise condition (NC) and/or a low signal indication is present for an intrinsic ventricular event sensed by the right ventricular sensing stage 58. By way of one or more embodiments, the signal quality analysis unit 96 may generate a noise condition and/or low signal indication signal and may provide it to an evaluation unit 98. In at least one embodiment, by way of right ventricular tip electrode 22, right ventricular ring electrode 24, right ventricular stimulation generator 56 and right ventricular sensing stage 58, right ventricular stimulation pulses may be delivered in a demand mode to the right ventricle 42 of heart 40.

In at least one embodiment, in the same way left ventricular tip electrode 30 and left ventricular ring electrode 32 may be connected to the left ventricular stimulation pulse generator 64 and the left ventricular sensing stage 66 that may be connected to signal quality analysis unit 96 of the control unit 54, and that may allow for stimulating a left ventricle 46 of heart 40.

By way of one or more embodiments, the outputs of the ventricular sensing states 58 and 66, i.e., sense signals comprising ventricular events, and of the signal quality analysis unit 96, i.e., noise condition and/or low signal indication signals, may be provided to the evaluation unit 98. In at least one embodiment, the evaluation unit 98 may evaluate the signals by detecting useable intervals in the sense signals in dependence of the noise condition and/or low signal indication signals. In embodiments of the invention, useable intervals may be intervals, which may be defined by two consecutive events that do not have a noise condition and/or a low signal indication. The evaluation unit 98, in one or more embodiments, may execute various monitoring and stimulation algorithms in parallel to monitor specific heart functional and rhythm disorders, e.g., bradycardia, asystole, high ventricular rate, or other heart functional or rhythm disorders and to treat the disorder, e.g., by stimulating the left ventricle 46 and/or the right ventricle 42 of heart 40. The evaluation unit 98, in at least one embodiment, may determine an average interval duration and an average rate of events from the useable intervals and may use these parameters to detect bradycardia, asystole, and/or high ventricular rate. If a functional disorder has been detected, in one or more embodiments, the evaluation unit 98 may execute an alternative monitoring and stimulation algorithm for the specific functional disorder which has been detected. In at least one embodiment, the alternative monitoring and stimulation algorithm may attempt to detect, whether the functional disorder was terminated. A termination may occur, in one or more embodiments, caused by stimulating the left ventricle 46 or right ventricle 42 of heart 40. The detection of termination and a stimulation adjusted to a detected functional disorder, in at least one embodiment, may also be integrated in one monitoring and stimulation algorithm.

According to at least one embodiment, triggering and inhibition of delivery of stimulation pulses to the right atrium, the right ventricle or the left ventricle may be controlled by control unit 54. The timing that schedules delivery of stimulation pulses if needed, in at least one embodiment, may be controlled by a number of intervals that at least partly may depend on a hemodynamic demand of a patient that may be sensed using an activity sensor 72 that may be connected to control unit 54. Activity sensor 72, in at least one embodiment, may allow for rate adaptive pacing wherein a pacing rate (the rate of consecutive ventricular stimulation pulses for a duration of consecutive atrial stimulation pulses) may depend on a physiological demand of a patient that may be sensed by a way of activity sensor 72.

In one or more embodiments, a clock 82 may allow recording of events and signals in association with time stamps that may enable a synchronous evaluation of signals at a later point of time.

By way of at least one embodiment, for the purpose of composition of a far-field right ventricular electrogram (RV EGM) and a far-field left-ventricular electrogram (LV EGM) a far-field right ventricular electrogram recording unit 76 and a far-field left ventricular recording unit 74, respectively, may be provided. The far-field right ventricular electrogram recording unit 76, in at least one embodiment, may be connected to a case electrode that may be formed by at least an electrically conducting part of case 12 of the heart stimulator 10 and to the RV coil electrode 34. The far-field left ventricular recording unit 74, in one or more embodiments, may also be connected to the case electrode formed by a case 12 of heart stimulator 10 and to the left ventricular coil electrode 38.

In at least one embodiment, the near-field electrogram in the right ventricle 42 may be measured between the RV-tip electrode 22 and RV-ring electrode 24. Preferably, in one or more embodiments, the far-field electrogram in the right ventricle 42 may be measured between the RV-coil electrode 34 and the device housing 12. Alternatively, in at least one embodiment, the far-field electrogram in the right ventricle 42 may be measured between the RV-ring electrode 24 and the device housing 12.

Likewise, in one or more embodiments, the near-field electrogram in the left ventricle 46 may be measured between the LV-tip electrode 30 and LV-ring electrode 32. Preferably, in at least one embodiment, the far-field electrogram in left ventricle may be measured between the LV-coil electrode 38 and the device housing 12. Alternatively, in embodiments of the invention, the far-field electrogram in the left ventricle 46 may be measured between the LV-ring electrode 32 and the device housing 12.

In at least one embodiment, preferably, the far-field electrogram in the right ventricle 42 and the left ventricle 46 may be minimally filtered and have wide bandwidth, e.g., with lower corner frequency 4 Hz and high corner frequency 128 Hz, whereas the near-field electrograms in the right ventricle 42 and the left ventricle 46 may be filtered with narrower bandwidth, e.g., with lower corner frequency 18 Hz and high corner frequency 40 Hz. Accordingly, in one or more embodiments, right and left far-field ventricular recording units 76 and 74 may each include a band pass filter with lower corner frequency 4 Hz and high corner frequency 128 Hz. Right ventricular sensing stage 58 and left ventricular sensing stage 66 for picking up near-field electrograms in the right ventricle 42 and the left ventricle 46, according to at least one embodiment, may each include band-pass filters with narrower bandwidth, e.g., with lower corner frequency 18 Hz and high corner frequency 40 Hz.

Both the far-field electrograms and the near-field electrograms, in one or more embodiments, may be used to detect events in the signals and to determine intervals and/or a corresponding a rate of events. In at least one embodiment, the signal quality analysis unit 96 may determine whether a noise condition (NC) and/or a low signal indication may be present for an intrinsic event sensed by the sensing stages 58, 66 and/or the far-field ventricular electrogram recording units 74, 76. The corresponding noise condition (NC) and/or low signal indication signal, in one or more embodiments, may be provided to the evaluation unit 98. In at least one embodiment, the evaluation unit 98 may evaluate the outputs of the sensing stages 58, 66 and the far-field ventricular electrogram recording units 74, 76 in dependence of the noise condition (NC) and/or low signal indication signal, i.e., determining an average interval duration and an average rate of events from the useable intervals and using these parameters to detect bradycardia, asystole, and/or high ventricular rate.

According to at least one embodiment of the invention, the heart monitor 10 may be an implantable device, used as a loop recorder, that detects QRS complexes using the subcutaneous electrodes 22, 24, 30, 32 as shown in FIG. 2 and FIG. 3. In one or more embodiments, the heart monitor 10 may combine different electrode measurements to create a combined signal and then may perform QRS detection on the combined signal. In at least one embodiment, the detected QRS events may be classified as ventricular sense events (VS) 102 or as invalid sensed events (VN) 104. A VS 102, in at least one embodiment, may be considered a VN 104 if it has an associated noise condition (NC) or low signal indication.

One or more embodiments of the invention describe a High Ventricular Rate (HVR) detection unit for use in the heart monitor 10, which may implement an algorithm with additional handling for invalid sensed events (VN) 104. The HVR noise handling mechanism, according to at least one embodiment, is described in the following sections for detection and termination respectively.

High Ventricular Rate Unit

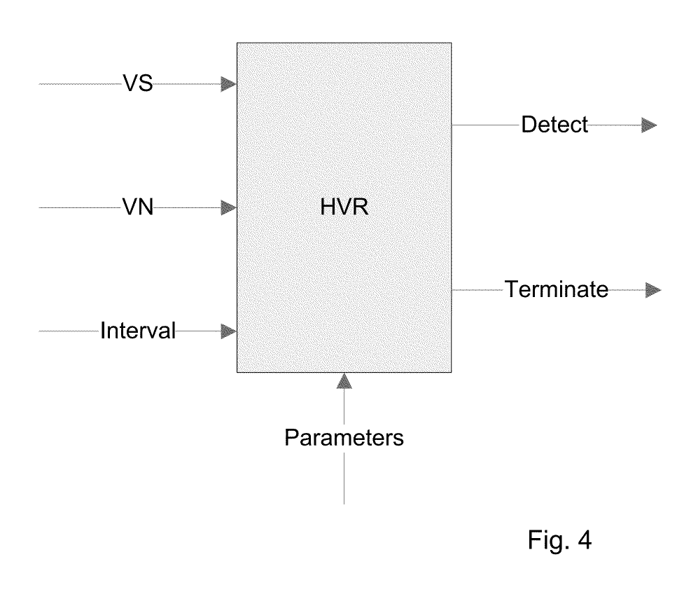

In at least one embodiment, HVR may use event type, interval and parameters as inputs. The following subsections describe such inputs. In one or more embodiments, the output of HVR unit may be detection or termination. FIG. 4 shows input and output interfaces of a high ventricular rate (HVR) according to one or more embodiments of the invention.

In at least one embodiment, input and output parameters of the High Ventricular Rate unit may include:

Event Types

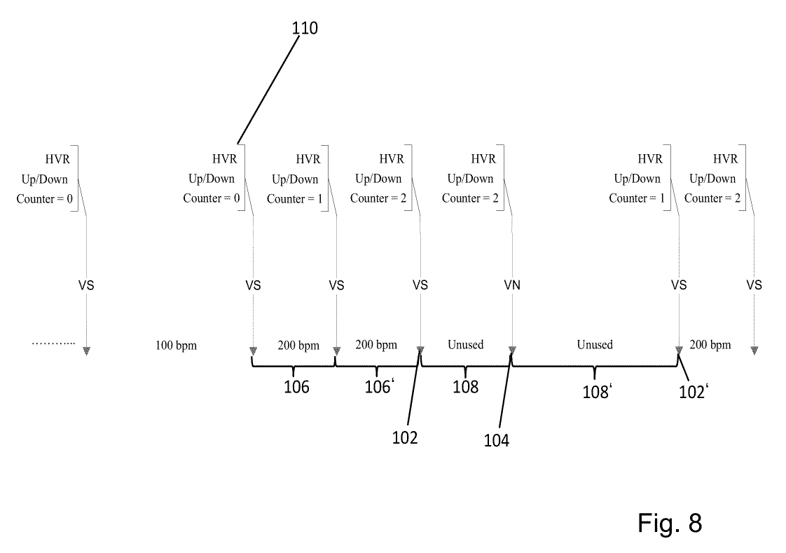

In one or more embodiments, the heart monitor 10 may include 2 event types, VS (used, respectively useable) 102 and VN (unused, respectively unuseable) 104, as shown in FIG. 8. When an implant software (ISW) executed on the control unit 54 calls HVR, in at least one embodiment, the implant software (ISW) may provide classification of the event type as either VS 102 or VN 104.

Intervals

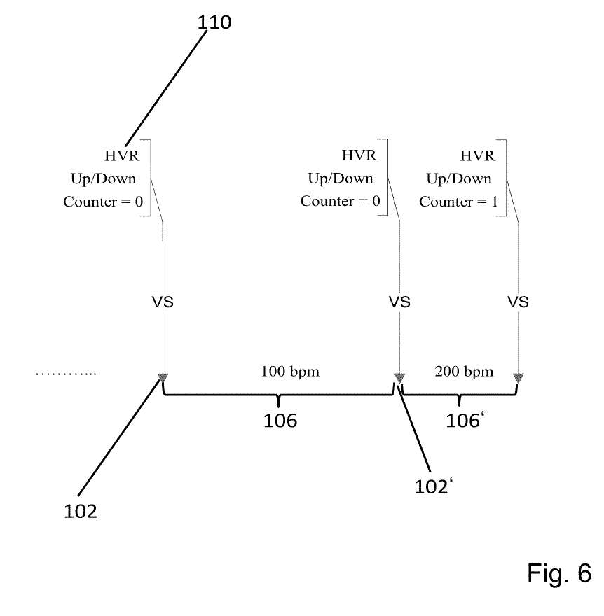

In one or more embodiments, a used interval 106 may be the interval duration measured between 2 consecutive VS 102, 102′ events, as shown in FIG. 6. In at least one embodiment, an interval may be considered unused 108, 108′ if at least 1 of the 2 consecutive events making up the interval is a VN 104, as shown in FIG. 8. In one or more embodiments, it is assumed that when the implant software (ISW) calls HVR, the implant software (ISW) may provide the most recent interval, if the interval is a used interval 106. The HVR unit, in at least one embodiment, may then test this interval to determine if it meets a high rate criterion. If the high rate criterion is met, in one or more embodiments, a high rate detection counter 110 may be incremented, as shown in FIG. 6. If the implant software (ISW) provides an unused interval 108′, in one or more embodiments, the high rate detection counter 102 may be decremented if the first event of the interval is a VN 104, as shown in FIG. 8. Unused intervals 108, 108′, in one or more embodiments, may not be tested for high rate conditions and may not affect termination.

Parameters

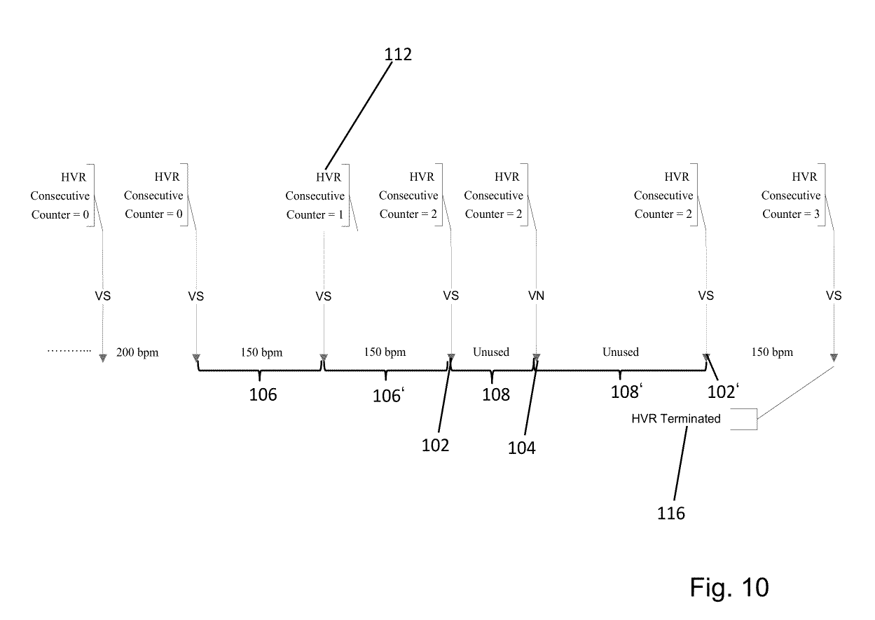

In at least one embodiment, high ventricular rate (HVR) may be detected when the high rate detection counter 110 exceeds a programmed threshold; as will be discussed below. In one or more embodiments, the high rate detection counter 110 may be incremented each time a rate above a programmed rate limit is detected. The programmed rate limit, in at least one embodiment, may be programmable in the range from 150 to 200 bpm in steps of 10 bpm, wherein the default value may be 180 bpm. Instead of incrementing the high rate detection counter 110 by exceeding the programmed rate limit, in one or more embodiments, the high rate detection counter 110 may also be incremented when a used interval exceeds a detection interval limit. In at least one embodiment, the detection interval limit may correspond to the programmed rate limit, i.e., the detection interval limit may be programmable in the range from 0.3 s to 0.4 s and may have a default value of 0.33 s. The rate may be the inverse to the interval. In one or more embodiments, the threshold for the high rate detection counter 110 may be programmable in the range from 4 to 16 in steps of 4, wherein the default value may be 8. Once HVR detects, in at least one embodiment, HVR may be declared and HVR may change mode to attempt to terminate HVR. In one or more embodiments, HVR may be terminated when the rate is below the programmed rate limit for a programmed number of consecutive used intervals 106. Unused intervals 108, in at least one embodiment, may play no role in termination and may not interrupt the calculation of consecutive used intervals, as shown in FIG. 10, and discussed further below. The programmed number of consecutive used intervals 106, in one or more embodiments, may be programmable in the range from 4 to 16 in steps of 1, wherein the default value may be 5.

High Ventricular Rate Algorithm

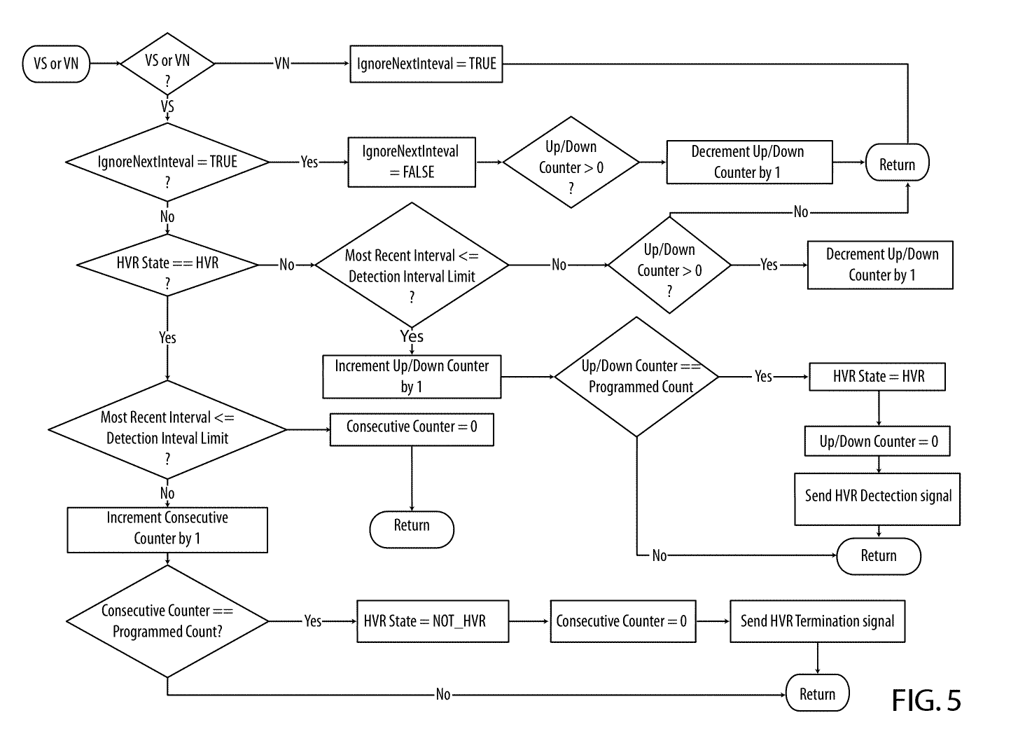

In at least one embodiment, high ventricular rate (HVR) may be detected when the high rate detection counter 110 exceeds the programmed threshold. In one or more embodiments, the high rate detection counter 110 may be incremented for used intervals 106 with a rate above the programmed rate threshold and decremented when unused intervals 108′ may be provided by the implant software (ISW). Once HVR detects, in at least one embodiment, then HVR may be declared and HVR may change mode to detect termination. HVR is terminated, in one or more embodiments, when the rate is below the programmed rate limit for a programmed number of consecutive used intervals 106. A detailed flow chart of the HVR algorithm is shown in FIG. 5.

FIG. 5 shows a flow chart of a HVR algorithm for detection and termination of a HVR according to one or more embodiments of the invention. According to at least one embodiment, the HVR algorithm presented in FIG. 5 may include one or more of the following steps:

200 Detect a QRS complex, i.e., a QRS event, in the combined electric signal.

210 Classify the QRS event as either ventricular sensed event VS 102 or invalid sensed event VN 104.

220 Set an ignore next interval flag to true, if QRS event is classified as VN 104.

230 Return to the start of the algorithm, i.e., step 200.

240 Check if the ignore next interval flag is set true.

250 Set the ignore next interval flag to false, if the ignore next interval flag was set as true.

260 Check if the high rate detection counter 110 is greater than 0.

270 Decrement the high rate detection counter 110 by 1, if the high rate detection counter 110 is greater than 0.

280 Check if a HVR state is detected, i.e., check if HVR state flag is true.

290 Check if the most recent interval is smaller than or equal to the detection interval limit.

300 Increment the high rate detection counter 110 by 1, if the most recent interval is smaller than or equal to the detection interval limit.

310 Check if the high rate detection counter 110 is equal to the programmed count.

320 Set the HVR state flag to true, if the high rate detection counter 110 is equal to the programmed count.

330 Set the high rate detection counter to 0, if the high rate detection counter 110 is equal to the programmed count.

340 Send a HVR detection signal, if the high rate detection counter 110 is equal to the programmed count.

In at least one embodiment, the HVR detection signal may be used for other devices, e.g., stimulators to perform stimulation according to, e.g., a HVR stimulation scheme, or to a drug injection device that may release drugs for treating the high ventricular rate.

350 Set a termination counter 114, i.e. consecutive counter, to 0, if the most recent interval is smaller than or equal to the detection interval limit.

360 Increment the termination counter 114 by 1, if the most recent interval is not smaller than or not equal to the detection interval limit.

370 Check if the termination counter 114 is equal to the programmed count.

380 Set a HVR state flag to false, i.e., set a HVR state to not HVR, if the termination counter 114 is equal to the programmed count.

390 Set the termination counter 114 to 0, if the termination counter 114 is equal to the programmed count.

400 Send a HVR termination signal, if the termination counter 114 is equal to the programmed count.

Detection

In at least one embodiment, the High ventricular rate (HVR) unit may use the high rate detection counter 110 for detection, which may operate as an up/down counter. Each used interval 106, in one or more embodiments, that may have an equivalent rate greater than or equal to the programmed rate limit, may increase the high rate detection counter 110. Each used interval 106, in one or more embodiment, that may have an equivalent rate below the programmed rate limit, may decrease the high rate counter 110. Each unused interval 108 and 108′, in one or more embodiments, may also decrease the high rate counter 110. This may prevent HVR detection from occurring when only a few good intervals 106 are present during long periods of noise. In at least one embodiment, the resulting used intervals 106 may have little relationship to each other in time in these circumstances. In one or more embodiments, the result may be a more specific HVR detection. When the high rate detection counter 110 reaches the programmed number of used intervals 106, in at least one embodiment, then HVR may be detected.

As shown in FIGS. 6-10, the most recent event is on the right and the oldest event is on the left. In at least one embodiment, the HVR rate limit may be programmed to 180 bpm, the number of used intervals 106 may be programmed to 3, and the consecutive number of used intervals 106 may be set to 3, as shown in FIGS. 6-10.

As shown in FIGS. 6-8, according to at least one embodiment, the high rate detection counter 110 used for detection is shown as HVR up/down counter.

Used Interval Handling

FIG. 6 shows two intervals of an exemplary evaluation of a high ventricular rate (HVR) according to one or more embodiments of the invention. As shown in FIG. 6, in one or more embodiments, a new used interval 106′ may be acquired, and then the HVR high rate detection counter 110 may be incremented. In at least one embodiment, the short interval 106′ may cause the HVR high rate detection counter 110 to increment with a rate limit that may be programmed to 180 bpm.

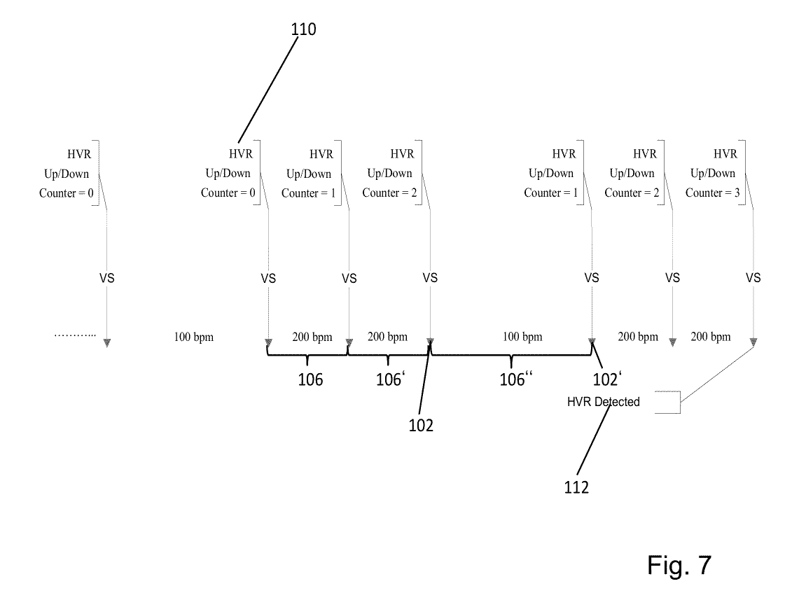

FIG. 7 shows a first exemplary evaluation of a high ventricular rate (HVR) with HVR detection according to one or more embodiments of the invention. As shown in FIG. 7, in one or more embodiments, the high rate detection counter 110 may be incremented twice due to 2 fast intervals 106, 106′, then may be decremented once (due to 1 slower interval 106″) and then may be incremented twice resulting in an HVR detection 112. In at least one embodiment, HVR may be detected with only used intervals 106 with a rate limit programmed to 180 bpm and the number of used intervals 106 may be programmed to 3.

Unused Interval Handling

According to one or more embodiments, intervals created using a VN 104 at the start of the interval 108′ may cause the high rate detection counter 110 to be decremented; as will be discussed below.

FIG. 8 shows a first exemplary evaluation of a high ventricular rate (HVR) without HVR detection according to one or more embodiments of the invention. As shown in FIG. 3, in one or more embodiments, the high rate detection counter 110 may be incremented twice due to 2 fast intervals 106, 106′, then may remain unchanged for an interval 108 created with a detected VN 104 and the high rate detection counter 110 may be decremented once for the following interval 108′ (due to 2 unused intervals 108, 108′) and then may be incremented once resulting in no HVR detection. In at least one embodiment, the HVR may not be detected with intermittent unused intervals 108, 108′ with a rate limit that may be programmed to 180 bpm and the number of used intervals 106 that may be programmed to 3.

In an alternative embodiment, the intervals 108, 108′ created using a VN 104 may cause the high rate detection counter 110 to remain unchanged. In this case, the high rate detection counter 110 may be incremented twice due to 2 fast intervals 106, 106′, then may remain unchanged for 2 intervals 108, 108′ (due to 2 unused intervals) and may then be incremented once resulting in HVR detection (not shown).

Termination

According to one or more embodiments, termination of HVR may be attempted following detection of HVR. In at least one embodiment, high ventricular rate (HVR) termination may use a termination counter 112, working as a consecutive counter. Each used interval 106 that has an equivalent rate less than the programmed rate limit, in at least one embodiment, may increase the termination counter 114. In one or more embodiments, each used interval 106 that has an equivalent rate greater than or equal to the programmed rate limit may clear the termination counter 114. When the termination counter 114 reaches the programmed number of consecutive used intervals 106, in at least one embodiment, then HVR may be terminated. In one or more embodiments, unused intervals 108 may play no role in HVR termination.

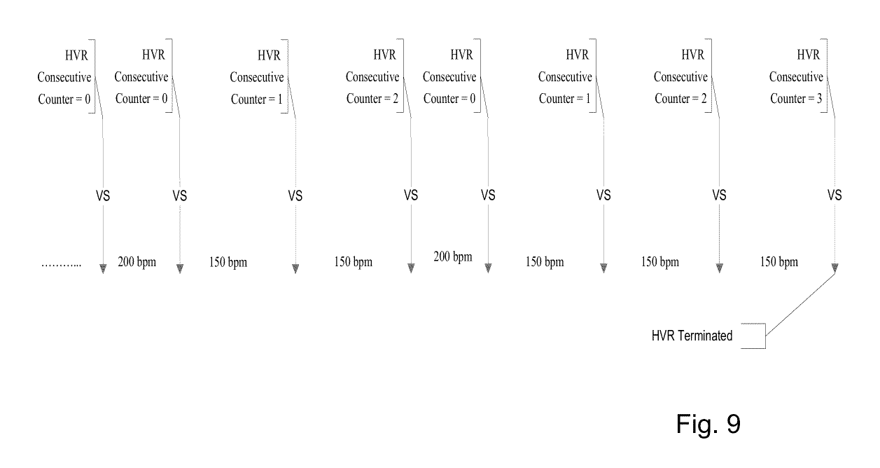

FIG. 9 shows a first exemplary evaluation of a high ventricular rate (HVR) with HVR termination according to one or more embodiments of the invention, and FIG. 10 shows a second exemplary evaluation of a high ventricular rate (HVR) with HVR termination according to one or more embodiments of the invention. As shown in FIGS. 9 and 10, in one or more embodiments, the termination counter 114, i.e., HVR consecutive counter, may be used for termination, as shown.

Used Interval Handling

As shown in FIG. 9, according to at least one embodiment, the termination counter 114 may be incremented twice due to 2 slow intervals 106, 106′, then may be reset to zero (due to 1 fast interval 106″) and then may be incremented 3 times, resulting in an HVR termination 116. In at least one embodiment, HVR may be terminated with only used intervals 106 with a rate limit that may be programmed to 180 bpm and the number of consecutive used intervals 106 that may be programmed to 3.

Unused Interval Handling

As shown in FIG. 10, in one or more embodiments, the termination counter 114 may be incremented twice due to 2 slow intervals 106, 106′, then may remain unchanged for 2 intervals 108, 108′ (due to 2 unused intervals 108, 108′) and then may be incremented once resulting in an HVR termination 116. In at least one embodiment, HVR may be terminated with intermittent unused intervals 108, 108′ with a rate limit that may be programmed to 180 bpm and the number of consecutive used intervals 106 that may be programmed to 3.

Brady and Asystole Unit

One or more embodiments of the invention describe the proposed Brady and Asystole detection unit for use in the heart monitor 10. In at least one embodiment, asystole may be defined as a ventricular interval greater than or equal to 3 seconds, and bradycardia may be defined as a decrease of heart rate by 30%, Brady Rate Drop, or a heart rate lower than 40 bpm that may be sustained longer than 10 seconds, Brady Rate Limit. In one or more embodiments, Brady rate drop, Brady rate limit and asystole have a noise handling mechanism as will be discussed below.

Used Intervals

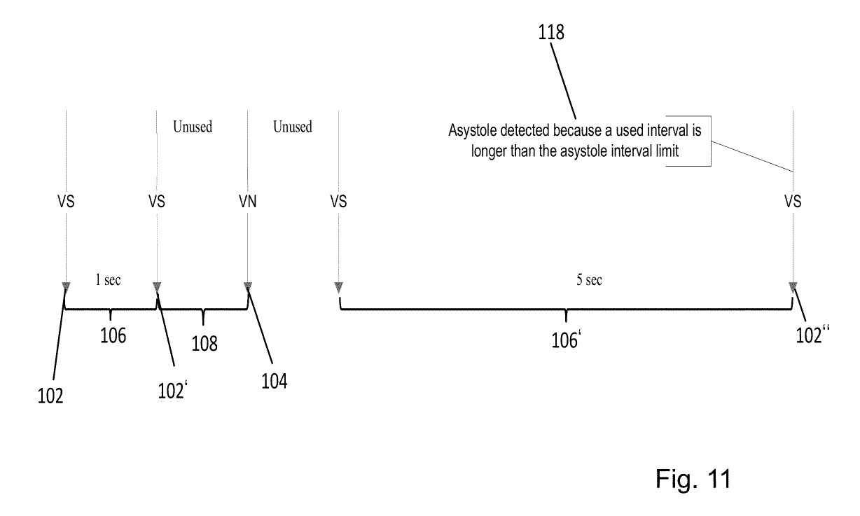

In one or more embodiments of the invention, the heart monitor 10 may operate with the 2 event types, VS (used) 102 and VN (unused) 104. In at least one embodiment, a used interval 106 may be the interval measured between 2 consecutive VS events 102, 102′, as shown in FIG. 11. In at least one embodiment, an interval 108 may be considered unused if at least 1 of the 2 consecutive events making up the interval is a VN 104, as shown in FIG. 11.

Asystole

According to one or more embodiments, asystole 118 may be detected if a single usable interval 106′ is longer than the programmed asystole interval limit, otherwise it may not be detected, as shown in FIG. 11. In at least one embodiment, the asystole interval limit may be programmed in the range from 2 to 10 seconds in steps of 1 second, wherein the default value is 3 seconds.

FIG. 11 shows an example of a detection of an asystole according to one or more embodiments of the invention. As shown in FIG. 11, in one or more embodiments, the most recent event is on the right and oldest event is on the left and the programmed asystole interval limit may be 3 seconds.

As also shown in FIG. 11, according to at least one embodiment, the asystole 118 may be detected on the VS 102″ concluding the most recent interval 106′ because the used interval 106′ may be longer than the programmed asystole interval limit.

Bradycardia

In one or more embodiments, bradycardia may be detected by either Brady rate limit or Brady rate drop, and may run independently and in parallel until either one of them detects bradycardia. In at least one embodiment, once either algorithm detects, then bradycardia may be declared and the bradycardia detection algorithm may change into a termination mode and may attempt to detect termination.

Brady Rate Limit

In one or more embodiments of the invention, Brady rate limit may be detected when the average rate is less than a programmed rate limit for a programmed duration. In at least one embodiment, the rate limit may be programmed in the range from 30 to 80 bpm in steps of 5 bpm. In one or more embodiments, the programmed duration may be programmed from 5 to 30 seconds in steps of 5 seconds, wherein the default values may be 40 bpm for 10 seconds.

Detection

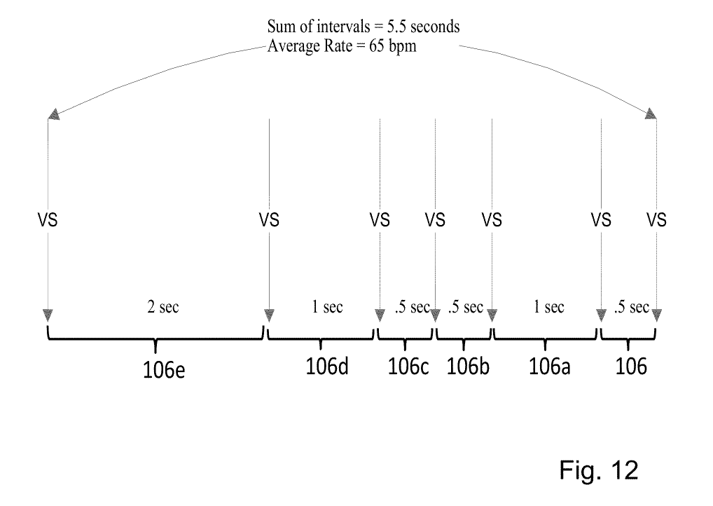

In one or more embodiments of the invention, used intervals 106, 106a, 106b, 106c, 106d, 106e may be summed starting with the newest used interval 106, going back in time, to find the minimum number of consecutive used intervals 106 whose sum may be greater than or equal to the programmed duration, as shown in FIG. 12. In at least one embodiment, the newest used interval 106 may always be included in the previously mentioned sum. This sum, according to at least one embodiment, may be updated on every used interval 106. If there are enough used intervals, in one or more embodiments, such that their sum is greater than or equal to the programmed duration, then the average of these intervals may be computed and then converted to a rate. If this rate is less than the programmed rate limit, in one or more embodiments, then Brady rate limit may be detected, otherwise Brady rate limit may not be detected. If the sum of all of the used intervals is less than programmed duration, in at least one embodiment, then Brady rate limit may not be detected. When Brady rate limit is initialized, in one or more embodiments, the number of usable intervals may be set to zero.

FIGS. 12-16 illustrate the most recent event on the right, and wherein events get older from right to left and the programmed asystole duration may be 5 seconds.

FIG. 12 shows a first exemplary evaluation of a Brady rate limit without detection of bradycardia according to one or more embodiments of the invention. As shown in FIG. 12, in one or more embodiments, the sum of the used intervals may be greater than the programmed duration, such that the average rate may be computed and compared to the programmed rate limit. In at least one embodiment, the average rate may be 65 bpm and therefore greater than the Brady rate limit, leading to no bradycardia detection.

Unused Interval Handling

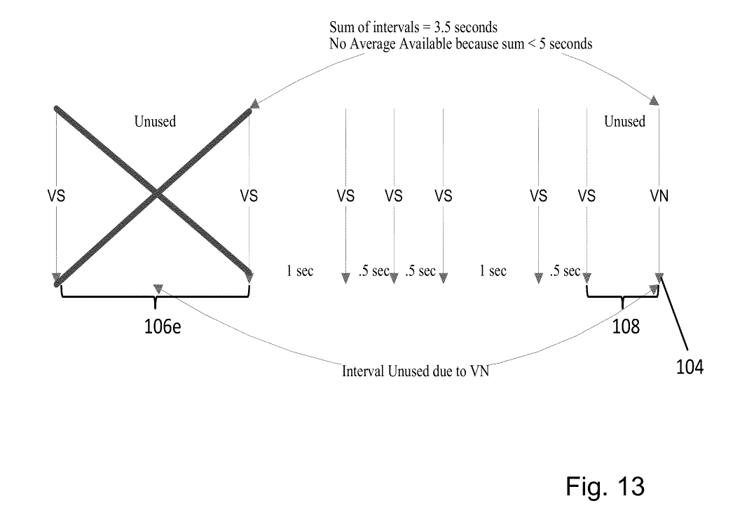

In one or more embodiments, following a VN 104, the Brady rate limit detection algorithm may remove the oldest used interval 106e from the previously used intervals to calculate a new used-interval-sum. FIG. 13 shows a second exemplary evaluation of a Brady rate limit with an interval sum below a programmed duration according to one or more embodiments of the invention. As shown in FIG. 13, in at least one embodiment, a VN 104 may occur which may lead to removal of the oldest used interval 106e. In addition, according to one or more embodiments, the newest interval 108 may not be added to the used-interval-sum because it may be unused. Therefore, in at least one embodiment, the VN 104 may cause the removal of 1 used interval.

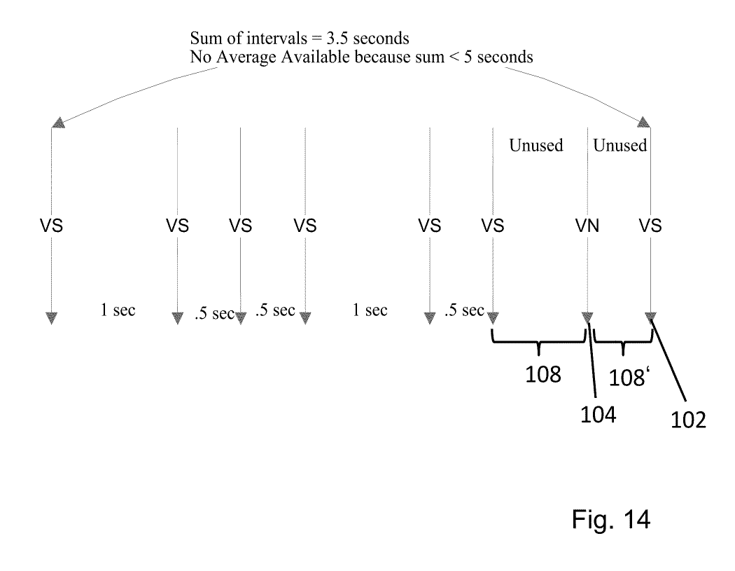

By way of at least one embodiment, a VS 102 following a VN 104 may not produce a usable interval, but an unused interval 108′, therefore the used-interval-sum may be unchanged, as shown in FIG. 14. FIG. 14 shows a third exemplary evaluation of a Brady rate limit with an interval sum below a programmed duration according to one or more embodiments of the invention. In at least one embodiment, the usable intervals may be the same in FIG. 13 and FIG. 14.

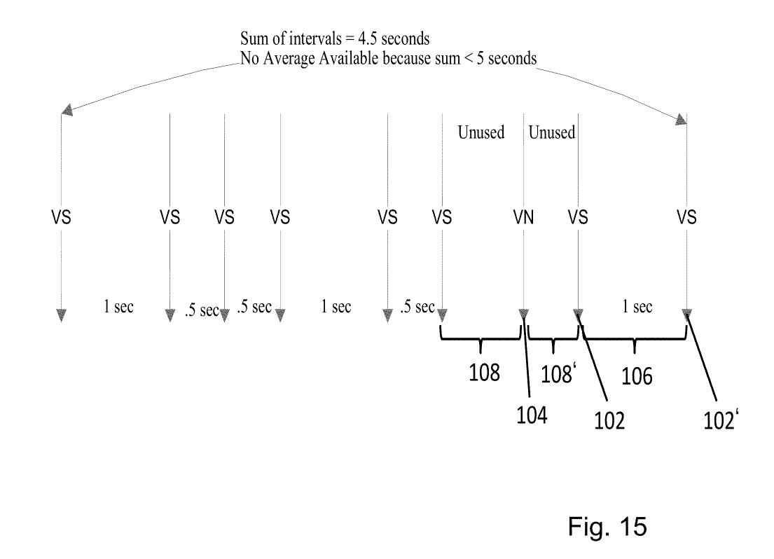

FIG. 15 shows a fourth exemplary evaluation of a Brady rate limit with an interval sum below a programmed duration according to one or more embodiments of the invention. As shown in FIG. 15, in one or more embodiments, a new used interval 106 may be acquired, then the used intervals may be summed and the unused intervals 108, 108′ may be ignored in the used-interval-sum. In at least one embodiment, the used-interval-sum may be less than the programmed duration, such that the average rate may not be compared to the programmed rate limit.

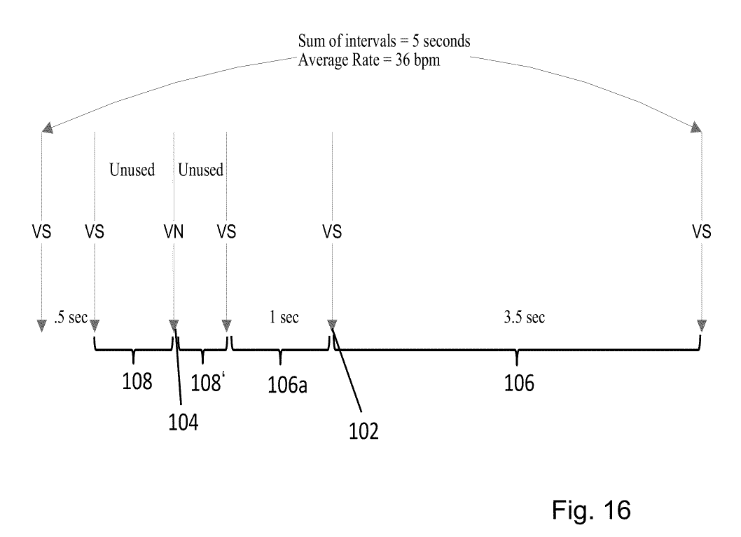

FIG. 16 shows a fifth exemplary evaluation of a Brady rate limit with an average rate of events indicative of bradycardia according to one or more embodiments of the invention. As shown in FIG. 16, in one or more embodiments, a new used interval 106 may be acquired, then the used intervals may be summed and the unused intervals 108, 108′ may be ignored in the used-interval-sum. In at least one embodiment, the used-interval-sum may be equal to the programmed duration, such that the average rate may be computed and compared to the programmed rate limit. According to at least one embodiment, the average rate may be 36 bpm, which is below the default rate limit of 40 bpm for the Brady rate limit resulting in a detection of bradycardia.

Brady Rate Drop

By way of one or more embodiments, Brady rate drop may be detected when the rate decreases by a programmed percentage. In at least one embodiment, the change in rate may be measured by comparing a pre-interval average 120 and a post-interval average 122. The number of intervals used in the pre-interval average 120, in one or more embodiments, may be programmed to 32, 48 or 64. In at least one embodiment, the number of intervals used in the post-interval average 122 may be programmed to 4, 8 or 16. According to at least one embodiment, the default values may be 48, 8 and 30% for the pre-interval number, wherein post-interval number and rate may decrease in percentage respectively.

Detection

By way of one or more embodiments, Brady rate drop may not be detected unless the total number of used intervals 106 is equal to the programmed pre-interval number plus the programmed post-interval number. In at least one embodiment, the number of used intervals may be set to zero upon initialization. For each new used interval 106, in one or more embodiments, the number of used intervals may be incremented as long as it does not exceed the pre-interval number plus post-interval number of used intervals, else the interval number equals the pre-interval number plus post-interval number.

In one or more embodiments of the invention, the post-interval average 122 may be calculated by taking the average of the most recent post-interval number of used intervals. The post-interval average 122, in at least one embodiment, may always be computed using the most recent used interval 106. The pre-interval average 120 may be computed using the previous consecutive pre-interval number of used intervals preceding the intervals used to calculate the post-interval average, according to one or more embodiments. If there are pre-interval number plus post-interval number of used intervals, in at least one embodiment, these averages may be updated on every used interval 106. Both of these averages 120, 122 may then be converted to a rate. In one or more embodiments, if the post-interval average rate is <(1−programmed rate decrease percentage)*pre-interval average rate, then Brady rate drop may be detected, else it may not be.

According to at least one embodiment, once Brady rate drop is detected, the number of usable intervals may be set to the programmed post-interval number. This effectively may remove all intervals used in the pre-interval-average 120 from further Brady rate drop detections.

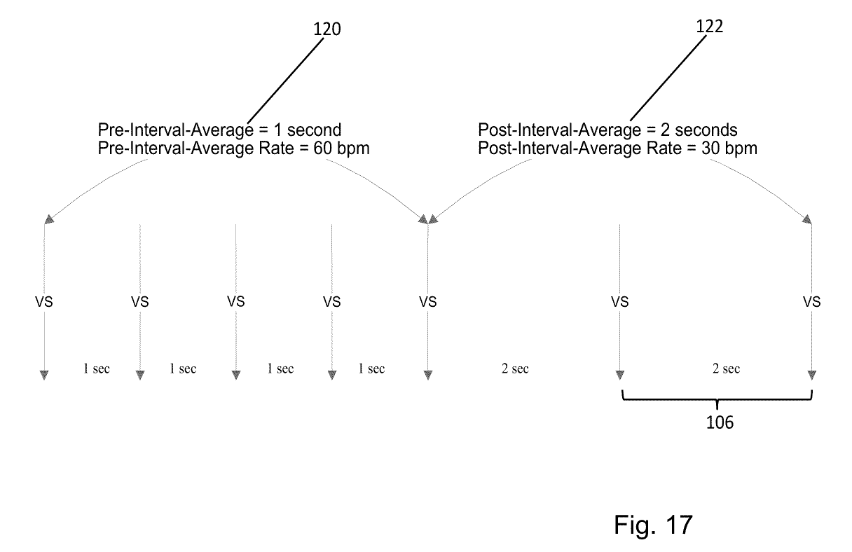

FIGS. 17-20 illustrate the most recent event on the right, and wherein events get older from right to left. In one or more embodiments, the pre-interval number and the post-interval number may be set to 4 and 2 respectively for FIGS. 17-20. In at least one embodiment, values of 4 and 2 may not be allowable settings in the implantable heart monitoring device 10.

FIG. 17 shows a first exemplary evaluation of a Brady rate drop with detection of bradycardia according to one or more embodiments of the invention. As shown in FIG. 17, in one or more embodiments, a new interval may be acquired thereby filling the pre-interval buffer, and then the pre-interval averages 120 and post-interval averages 122 may be computed. In at least one embodiment, the rates derived from the pre-interval averages 120 and post-interval averages 122 may lead to a pre-interval-average rate of 60 bpm and a post-interval-average rate of 30 bpm, a 50% rate decrease, which may result in a detection of bradycardia for the default rate decrease percentage of 30%.

Unused Interval Handling

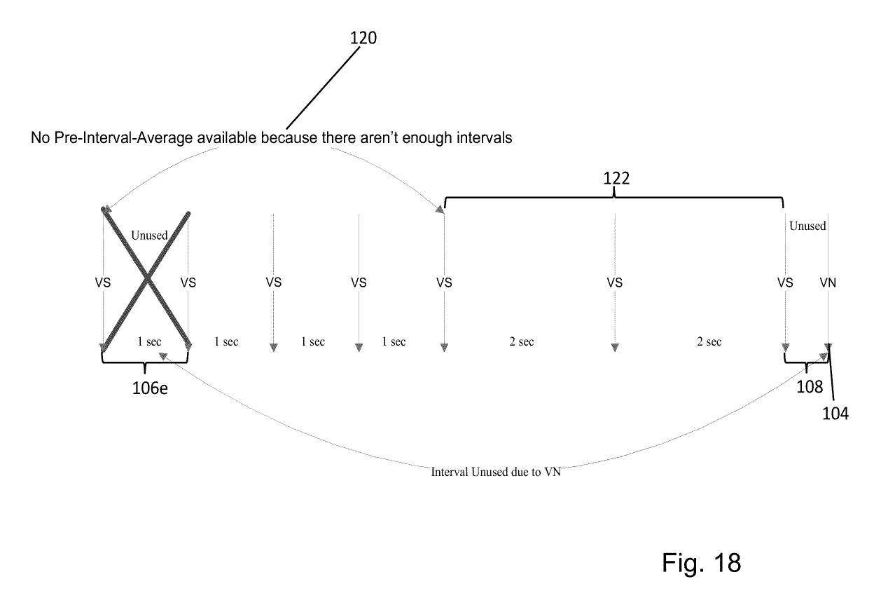

By way of at least one embodiment, following a VN 104, the Brady rate drop algorithm may remove the oldest used interval 106e from the previously used intervals and may decrement the usable interval count by 1. FIG. 18 shows a second exemplary evaluation of a Brady rate drop with a too small number of intervals according to one or more embodiments of the invention. As shown in FIG. 18, in one or more embodiments, a VN 104 may occur which may lead to removal of the oldest used interval 106e. In addition, in at least one embodiment, the newest interval 108 may not be added to the usable intervals because it may be unused. Following the VN 104, in one or more embodiments, the number of intervals may be less than the programmed pre-interval number and the post-interval number. In at least one embodiment, no pre-internal-average may be available because there may not be enough intervals.

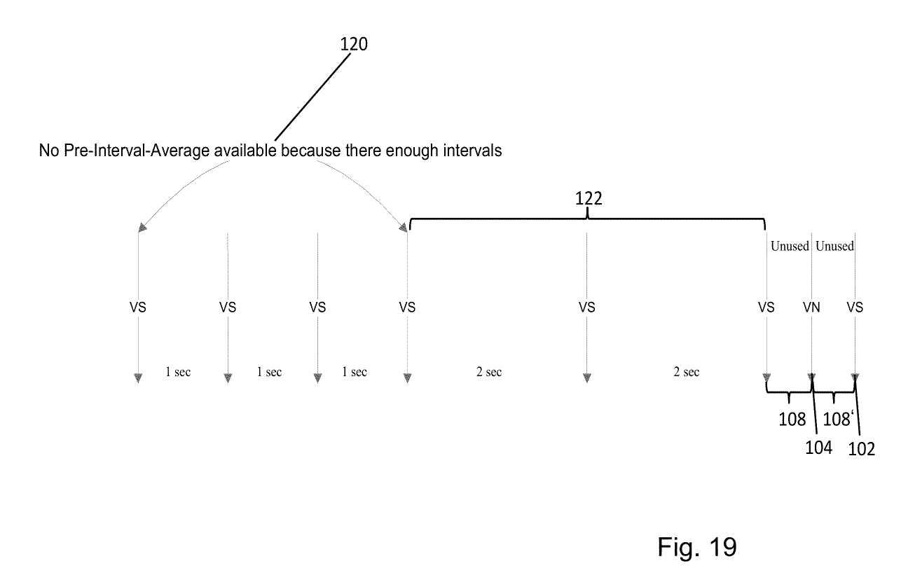

FIG. 19 shows a third exemplary evaluation of a Brady rate drop with a too small number of intervals according to one or more embodiments of the invention. As shown in FIG. 19, a VS 102 following a VN 104 may not produce a usable interval, but an unused interval 108′, therefore the usable interval count may be unchanged. In at least one embodiment, the usable intervals may be the same in FIG. 18 and FIG. 19. In one or more embodiments, no pre-internal-average may be available because there may not be enough intervals.

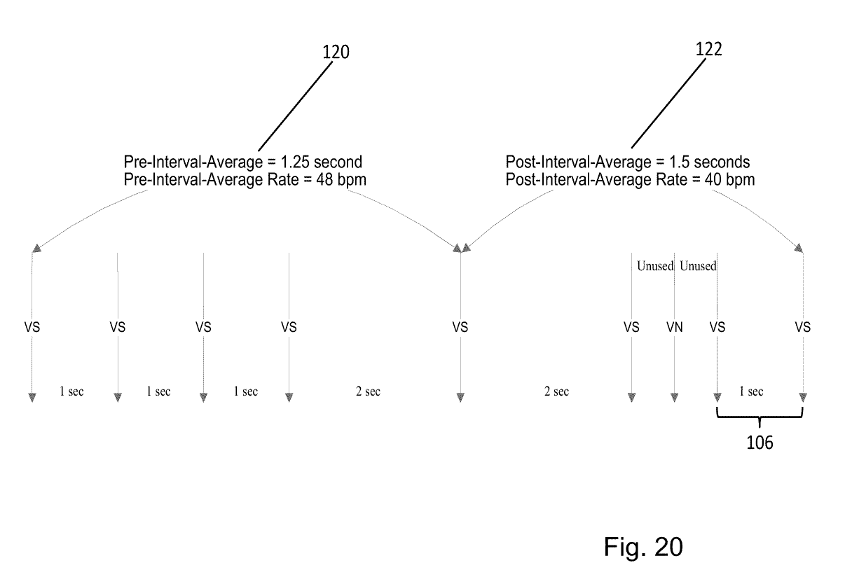

FIG. 20 shows a fourth exemplary evaluation of a Brady rate drop without detection of bradycardia according to one or more embodiments of the invention. As shown in FIG. 20, a new used interval 106 may be acquired. Then, in at least one embodiment, the pre-interval averages 120 and post-interval averages 122 may be computed. By way of at least one embodiment, the usable interval count may equal the programmed pre-interval number plus the post-interval number, such that the average rates may be calculated and compared to see if Brady Rate Drop may be detected. In at least one embodiment as shown in FIG. 20, no Brady rate drop may be detected, as the rate decrease percentage is below 30%.

Termination

According to at least one embodiment of the invention, once bradycardia may be detected by either Brady rate limit or Brady rate drop, the algorithm may change into the termination mode and may attempt to detect termination. In one or more embodiments, bradycardia may be terminated after a bradycardia termination counter reaches 10. The bradycardia termination counter, in embodiments of the invention, may be initialized to zero on detection of Brady rate limit or Brady rate drop. The termination count, in one or more embodiments, may be incremented for each used interval shorter than the interval equivalent of the programmed rate limit. In at least one embodiment, the programmed rate limit used for Brady rate limit detection may be the same rate limit used for termination of Brady rate limit and Brady rate drop. By way of one or more embodiments, the termination count may be reset to zero for each used interval that may be longer than or equal to the interval equivalent of the programmed rate limit. The termination counter, in at least one embodiment, may be decremented for each VN 104, if the termination count is greater than zero; otherwise the termination counter remains zero.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. An implantable hear monitor comprising:
a sensing unit configured to be connected to at least one electrode configured to pick up electric potentials,
wherein the sensing unit is configured to
process electrical signals corresponding to said electric potentials,
detect signal components representing a myocardial contraction, and
generate sense signals representing detected events corresponding to myocardial contractions;
a signal quality analysis unit configured to
determine whether a noise condition (NC) and/or a low signal indication is present for an event, and
generate a noise condition and/or low signal indication signal; and
an evaluation unit connected to said sensing unit and said signal quality analysis unit,
wherein said evaluation unit is configured to
evaluate sense signals and noise condition and/or low signal indication signals,
treat the detected events as non-valid if a noise condition (NC) and/or low signal indication signal is present for that event, and
use only intervals defined by two consecutive events which do not have a noise condition (NC) and/or a low signal indication to determine a rate of events of the sense signal.

2. The implantable heart monitoring device according to claim 1, wherein the sensing unit is a ventricular sensing unit configured to detect ventricular events.

3. The implantable heart monitoring device according to claim 2, wherein the evaluation unit is further configured to detect a high ventricular rate, if a high rate detection counter exceeds a predetermined threshold, and increment the high rate detection counter each time the determined rate of events in a used interval exceeds a predetermined threshold of a high ventricular rate detection limit.

4. The implantable heart monitoring device according to claim 3, wherein the evaluation unit is further configured to decrement the high rate detection counter each time the determined rate of events in a used interval is below the predetermined threshold of the high ventricular rate detection limit.

5. The implantable heart monitoring device according to claim 3, wherein the evaluation unit is further configured to decrement the high rate detection counter each time an event has a noise condition (NC) and/or a low signal indication.

6. The implantable heart monitoring device according to claim 3, wherein the evaluation unit is further configured to detect a termination of the high ventricular rate, if the high ventricular rate was detected and a termination counter exceeds a predetermined threshold, and increment the termination counter each time the determined rate of events in a used interval is below a predetermined threshold of a high ventricular rate detection limit.

7. The implantable heart monitoring device according to claim 6, wherein the evaluation unit is further configured to set the termination counter to zero each time the determined rate of events in a used interval exceeds the predetermined threshold of a high ventricular rate detection limit.

8. The implantable heart monitoring device according to at least one of the claim 1, wherein the evaluation unit is further configured to detect an asystole if a used interval is longer than a predetermined asystole interval limit.

9. The implantable heart monitoring device according to at least one of the claim 1, wherein the evaluation unit is further configured to detect bradycardia, if an average rate of events is less than a predetermined Brady rate limit for a predetermined bradycardia duration.

10. The implantable heart monitoring device according to claim 9, wherein the evaluation unit is further configured to find a minimum number of used intervals whose sum exceeds the predetermined bradycardia duration, determine an average duration of the summed used intervals, convert the average duration into an average rate of events, and determine if the average rate of events is below the predetermined Brady rate limit.

11. The implantable heart monitoring device according to claim 10, wherein the evaluation unit is further configured to add the newest used interval to the minimum number of used intervals when in use, and if the sum of the used intervals without the oldest interval exceeds the predetermined bradycardia duration, remove the oldest used interval from the minimum number of used intervals.

12. The implantable heart monitoring device according to claim 10, wherein the evaluation unit is further configured to remove the oldest used interval from the minimum number of used intervals, if an event has a noise condition (NC) and/or a low signal indication.

13. The implantable heart monitoring device according to claim 1, wherein the evaluation unit is further configured to detect bradycardia, if an average rate of events decreases by a predetermined percentage threshold, and wherein a change in the average rate of events is determined by comparing a pre-interval average comprising a predetermined pre-interval number of used intervals and a post-interval average comprising a predetermined post-interval number of used intervals.

14. The implantable heart monitoring device according to claim 8, wherein the evaluation unit is further configured to detect a termination of bradycardia, if bradycardia was detected and a predetermined bradycardia termination counter exceeds a predetermined threshold, wherein the bradycardia termination counter is incremented for each used interval shorter than the interval equivalent of the predetermined Brady rate limit, and wherein, if the bradycardia termination counter is greater than zero, the bradycardia termination counter is decremented for each event which has a noise condition (NC) and/or a low signal indication.

15. The implantable heart monitoring device according to claim 14, wherein the evaluation unit is further configured to set the bradycardia termination counter to zero for each used interval that is longer than or equal to the interval equivalent of the predetermined Brady rate limit.

16. The implantable heart monitoring device according to claim 8, wherein the evaluation unit is further configured to set the number of usable intervals to 0 and remove all the used intervals, if an event has a noise condition (NC) and/or a low signal indication.
